(19) United States
(12) Patent Application Publication
(10) Pub. No.: US 2020/0000842 A1
(43) Pub. Date: Jan. 2, 2020

(54) COMPOSITION AND METHOD FOR IMPROVING QOL WITH MOLECULAR HYDROGEN IN CANCER- HAVING SUBJECT

(71) Applicant: MiZ Company Limited, Kanagawa (JP)

(72) Inventors: Fumitake Satoh, Kanagawa (JP); Shinichi Hirano, Kanagawa (JP); Ryosuke Kurokawa, Kanagawa (JP)

(21) Appl. No.: 16/456,085

(22) Filed: Jun. 28, 2019

(30) Foreign Application Priority Data
Jun. 29, 2018 (JP) 2018-124626

(51) Int. Cl. A61K 33/00; A61K 9/00; A61P 35/00

(52) U.S. Cl. CPC A61K 33/00; A61K 9/0019; A61P 35/00; A61K 9/0053; A61K 9/007

(57) ABSTRACT

The present application provides: a composition for improvement of QOL including improvement, suppression or reduction of at least one symptom attributed to cancer selected from the group consisting of cancerous pain, decreased appetite, insomnia, physical fatigue, and poor complexion or coloring in a subject, the composition being characterized by comprising molecular hydrogen as an effective ingredient; and a method for improving QOL in a subject with cancer, comprising administering the composition to the subject.

### FIELD OF THE INVENTION

The present invention relates to a composition for improvement of QOL including improvement, suppression or reduction of symptoms such as cancerous pain in a subject with cancer, the composition comprising molecular hydrogen (also referred to as hydrogen gas, gaseous hydrogen or hydrogen molecules) as an effective ingredient.

The present invention also relates to a method for improving QOL including improvement, suppression or reduction of symptoms such as cancerous pain in a subject, the method comprising administering the composition to the subject.

### BACKGROUND ART

Pain caused by cancer (hereinafter, referred to “cancerous pain”) in a cancer patient, particularly a terminal cancer patient, significantly degrades the quality of life (QOL) of the patient because chronic and intense pain persists, and normal pain relievers are not effective against the pain.

For treatment of such cancerous pain, an opioid such as morphine is used. The opioid is a medical narcotic drug having a high pain-relieving effect, and blocks transmission of pain from the spinal cord to the brain by binding to opioid receptors present in the spinal cord and the brain. Control of the dosage, the dose regimen and the like of the opioid by the doctor is absolutely necessary because the opioid itself causes drug dependence, and the opioid has side effects such as drowsiness, delirium/hallucination, respiratory depression, dry mouth, itching sensation, urination disorder, myoclonus, hyperpathia and side effects affecting the cardiovascular system.

As described in detail below, it is shown that in the present invention, a composition comprising molecular hydrogen as an effective ingredient is effective for improvement, suppression or reduction of symptoms such as cancerous pain in a subject with cancer.

Molecular hydrogen is said to protect against disorders from in vivo oxidative stress caused by reactive oxygen species (ROS), and there are some documents regarding a relationship between carcinogenesis and oxidative stress, and potentiality of use of molecular hydrogen for treatment of cancer (Patent Documents 1 to 3, and Non-Patent Documents 1 to 3). However, molecular hydrogen is not reported to improve, suppress or reduce symptoms such as cancerous pain.

### CITATION LIST

### Patent Document

[Patent Document 1] Japanese Patent Laid-Open No. 2007-254435

[Patent Document 2] Japanese Patent Laid-Open No. 2016-060732

[Patent Document 3] US 2015/0297514 A1

### Non-Patent Document

[Non-Patent Document 1] Shigeo Ota, Journal of the Japanese Biochemical Society 87(1): 82-90 (2015)

[Non-Patent Document 2] Takaaki Akaike et al. (edition), Experimental Medicine 36(5) (special issue): 161-170, 2018, Yodosha Co., Ltd. (Tokyo, Japan)

[Non-Patent Document 3] Toshikazu Yoshikawa (editorial supervision), Oxidative Stress in Medicine, second revised edition, Nos. 169 to 175, pp. 389-395, Shindan To Chiryosha, Inc. (Tokyo, Japan)

### SUMMARY OF THE INVENTION

### Technical Problem

It is an object of the present invention to provide a safer substance which enables improvement of QOL of a cancer patient, particularly improvement, suppression or reduction of symptoms such as cancerous pain in a cancer patient.

Pain caused by cancer spreading to surrounding tissues accounts for about 70% of cancerous pain, and it is said that inflammation of tissues, compression of nerve and the like result in occurrence of severe pain. Pain can be suppressed by an opioid (narcotic pain reliever) such as morphine, but as described above, the opioid causes drug dependence, and has side effects.

A pharmaceutical agent having higher safety may be able to contribute to further improvement of QOL of a cancer patient.

### Solution to Problem

The present inventors have extensively conducted studies, and resultantly found molecular hydrogen as a substance which enables improvement of QOL of a subject including improvement, suppression or reduction of symptoms such as cancerous pain. This finding is very surprising, and has not been reported yet.

Thus, the present invention includes the following features.

(1) A composition for improvement of QOL including improvement, suppression or reduction of at least one symptom attributed to cancer selected from the group consisting of cancerous pain, decreased appetite, insomnia, physical fatigue, and poor complexion or coloring in a subject with cancer, the composition comprising molecular hydrogen as an effective ingredient.

(2) The composition according to the above (1), wherein the composition is in the form of a hydrogen gas-containing gas and/or a hydrogen-dissolved liquid.

(3) The composition according to the above (2), wherein a hydrogen concentration of the hydrogen gas-containing gas is 0.5 to 18.5% by volume.

(4) The composition according to the above (2), wherein a hydrogen concentration of the hydrogen-dissolved liquid is 1 to 12 ppm.

(5) The composition according to any of the above (1) to (4), wherein the composition is administered to the subject by pulmonary administration, intravenous administration or oral administration.

(6) The composition according to the above (5), wherein the pulmonary administration is performed in an environment at atmospheric pressure, or in an environment at a high pressure of 1.02 to 7.0 atm.

(7) The composition according to any of the above (1) to (6), wherein the composition is prepared in situ using a hydrogen gas producing apparatus, a hydrogen water producing apparatus or a hydrogen gas adding apparatus, in administration of the composition to the subject.

(8) The composition according to any of the above (1) to (7), wherein the subject is a human.

(9) The composition according to any of the above (1) to (8), wherein the subject has terminal cancer.

(10) The composition according to any of the above (1) to (9), wherein the composition further has cancer cell growth-suppressive action and life-extending action.

(11) A method for improving QOL in a subject with cancer, the method comprising administering the composition according to any of the above (1) to (10) to the subject with cancer.

### Effect of the Invention

According to the present invention, symptoms such as cancerous pain are suppressed or reduced by administering molecular hydrogen to a cancer patient (for example terminal cancer patient), and the molecular hydrogen itself is known to have no side effect, so that QOL of the cancer patient is significantly improved. The molecular hydrogen is distributed to tissues including the tissues of the brain by diffusion mainly due to a property specific to gaseous molecules, and part of the molecular hydrogen is distributed to all parts of the body through blood flow, so that it is possible to reduce inflammation of tissues affected by cancer cells, suppress growth and metastasis of cancer cells, and suppress or reduce symptoms such as chronic and intense cancerous pain associated with cancer.

### BRIEF DESCRIPTION OF THE DRAWINGS

FIG. 1 shows a time-dependent change of the blood level of pancreatic cancer marker CA19-9 during about 8 months after the start of inhalation of hydrogen gas in a pancreatic cancer patient in Case 2 in Example 1.

### MODES FOR CARRYING OUT THE INVENTION

The present invention will be described in further detail.

### 1. Cancer and Cancerous Pain

The term “cancer” as used herein is intended to include malignant tumors, carcinomas and sarcomas.

Examples of cancer include, but are not limited to, gastric cancer, colorectal cancer, liver cancer, biliary tract cancer, renal cancer, bladder cancer, lung cancer, pancreatic cancer, esophageal cancer, breast cancer, cervical cancer, uterine cancer, ovarian cancer, brain tumor, laryngeal cancer, maxillary cancer, oral cavity cancer, lip cancer, thyroid cancer, cutaneous cancer, malignant melanoma, bone tumor, bone sarcoma, soft tissue tumor, angiosarcoma, pediatric solid tumor, leukemia and lymphoma.

The term “terminal cancer” as used herein refers to stage 3 cancer and stage 4 cancer.

The term “cancerous pain” as used herein refers to pain associated with cancer in a cancer patient such as a terminal cancer patient. The cancerous pain includes pain caused by cancer spreading to surrounding tissues through metastasis, and in this case, severe pain occurs due to inflammation of tissues, compression of nerve and the like. This results in occurrence of symptoms such that a normal pain reliever becomes less effective due to increased sensitivity to pain, pulse and breathing rates are increased, the blood pressure rises, the appetite is lost, and it becomes difficult to sleep. Thus, QOL of the patient is significantly degraded.

2. Composition for Suppression or Reduction of Cancerous Pain which Includes Molecular Hydrogen.

A first aspect of the present invention provides a composition for improvement of QOL including improvement, suppression or reduction of at least one symptom attributed to cancer selected from the group consisting of cancerous pain, decreased appetite, insomnia, physical fatigue, and poor complexion or coloring in a subject with cancer, the composition comprising molecular hydrogen as an effective ingredient.

Herein, the term “hydrogen” which is an effective ingredient of the composition of the present invention is molecular hydrogen (that is, gaseous hydrogen), and is sometimes referred to simply as “hydrogen” or “hydrogen gas”. In addition, the term “hydrogen” as used herein refers to hydrogen represented by the molecular formula of H₂, D₂ (deuterium) or HD (hydrogen deuteride), or a mixed gas thereof. D₂ is expensive, and is known to have a superoxide scavenging effect higher than that of H₂. The hydrogen usable in the present invention is H₂, D₂ (deuterium), HD (hydrogen deuteride), or a mixed gas thereof, preferably H₂. Alternatively, D₂ and/or HD may be used in place of H₂ or in combination with H₂.

A preferred form of the composition of the present invention is a form of a hydrogen gas-containing gas and/or a hydrogen-dissolved liquid.

The hydrogen gas-containing gas is preferably air containing hydrogen gas, or a mixed gas containing hydrogen gas and oxygen gas. The concentration of hydrogen gas in the hydrogen gas-containing gas is more than zero (0) and not more than 18.5% by volume, for example 0.5 to 18.5% by volume, preferably 1 to 10% by volume, for example 2 to 10% by volume, 2 to 8% by volume, 2 to 7% by volume, 2 to 6% by volume, 3 to 10% by volume, 3 to 8% by volume, 3 to 7% by volume, 3 to 6% by volume, 4 to 10% by volume, 4 to 8% by volume, 4 to 7% by volume, 4 to 6% by volume, 4 to 5% by volume, 5 to 10% by volume, 5 to 8% by volume, 6 to 8% by volume or 6 to 7% by volume, more preferably 5 to 10% by volume, for example 5 to 8% by volume, 6 to 8% by volume or 6 to 7% by volume. In the present invention, the cancerous pain-suppressive effect and the antitumor effect tend to be enhanced as the hydrogen gas concentration increases below the explosion limit.

Since hydrogen is a combustible and explosive gas, it is preferable to administer hydrogen to a subject such as a human with the hydrogen incorporated into the composition of the present invention under safe conditions for the subject in treatment of cancerous pain.

When the gas other than hydrogen gas is air, the concentration of the air is in the range of, for example, 81.5 to 99.5% by volume.

When the gas other than hydrogen gas is a gas containing oxygen gas, the concentration of the oxygen gas is in the range of, for example, 21 to 99.5% by volume.

Nitrogen gas may be present as another main gas. A gas such as carbon dioxide which is a gas present in the air may be present in an amount equivalent to the abundance in the air.

The hydrogen-dissolved liquid is specifically an aqueous liquid (for example a biologically applicable liquid) in which hydrogen gas is dissolved, and here, examples of the aqueous liquid include, but are not limited to, water (for example purified water and sterilized water), physiological saline, buffer solutions (for example, buffer solutions having a pH of 4 to 7.4), ethanol-containing water (for example, ethanol content: 0.1 to 2% by volume), drip-feed solutions, infusion solutions, injection solutions and beverages. The hydrogen concentration of the hydrogen-dissolved liquid is not limited, and is, for example, 1 to 12 ppm, 1 to 10 ppm, preferably 1.2 to 10 ppm, 1.2 to 8 ppm, for example 1.5 to 10 ppm, 1.5 to 7 ppm, 1.5 to 5 ppm, 2 to 10 ppm, 2 to 8 ppm, 2 to 7 ppm, 2 to 6 ppm, 2 to 5 ppm, 3 to 10 ppm, 3 to 8 ppm, 3 to 7 ppm, 4 to 10 ppm, 4 to 8 ppm, 5 to 10 ppm, 5 to 8 ppm, preferably 3 to 10 ppm, 3 to 8 ppm, 3 to 7 ppm, 4 to 10 ppm, 4 to 8 ppm, 5 to 10 ppm, or 5 to 8 ppm. In the present invention, the cancerous pain-suppressive effect and the antitumor effect tend to be enhanced as the concentration of hydrogen dissolved below the explosion limit increases.

A pharmaceutical product for treating cancer may be added to the hydrogen-dissolved liquid. Alternatively, the pharmaceutical product may be administered separately from administration of the hydrogen-dissolved liquid or the hydrogen gas-containing gas. Examples of the pharmaceutical product include, but are not limited to, chemotherapeutic agents, molecular target agents (for example antibody preparations) and immunotherapeutic agents (for example immune checkpoint inhibitors).

Examples of the chemotherapeutic agents include carboplatin, cyclophosphamide, cisplatin, docetaxel, nedaplatin, paclitaxel, pirarubicin, fluorouracil, bleomycin, mitomycin C, aclarubicin, ifosfamide, irinotecan, etoposide, erlotinib, gemcitabine, epirubicin, eribulin, goserelin, cytarabine, dexamethasone, doxorubicin, mitoxantrone, methotrexate, leuprorelin, vindesine, aclarubicin, oxaliplatin, nimustine, interferon a, teceleukin, temsirolimus, busulfan and melphalan.

Examples of the molecular target agents include antibodies such as cetuximab, bevacizumab, veltuzumab, lapatinib, trastuzumab, panitumumab and axitinib.

Examples of immune checkpoint inhibitors include PD-1 antibodies and PD-L1 antibodies. T cells which express PD-1 on the surface are produced for attacking cancer cells, and for counteracting the attack, the cancer cells produce PD-L1 to evade the attack by T cells. The above-described antibodies make it easier for T cells to attack cancer cells by binding to PD-1 and PD-L1.

Alternatively, the hydrogen-dissolved liquid may contain an opioid pain reliever (for example morphine, tramadol, oxycodone, fentanyl, tapentadol or methadone) or a non-opioid pain reliever (for example NSAIDs or acetaminophen), and in such a case, it may be possible to more effectively suppress or reduce intense cancerous pain by decreasing the dosage of the opioid or administering the hydrogen-dissolved liquid in combination with the non-opioid pain reliever. Alternatively, the hydrogen-dissolved liquid or the hydrogen gas-containing gas and the opioid pain reliever may be separately administered to the subject.

The hydrogen gas-containing gas or the hydrogen-dissolved liquid is blended with, for example, air or oxygen gas or a biologically applicable liquid in such a manner that a predetermined hydrogen gas concentration as shown above is obtained. Thereafter, the hydrogen gas-containing gas or the hydrogen-dissolved liquid is added into, for example, a pressure-resistant vessel (for example, a metallic cylinder (for example a stainless cylinder), an aluminum can, preferably a pressure-resistant plastic bottle with the inside laminated with an aluminum film (for example a pressure-resistant PET bottle) and a plastic bag, an aluminum bag). Aluminum has a property of being hardly permeable to hydrogen molecules. Alternatively, the hydrogen gas-containing gas or the hydrogen-dissolved liquid may be prepared in situ using an apparatus such as a hydrogen gas producing apparatus, a hydrogen water producing apparatus or a hydrogen gas adding apparatus, for example a known or commercially available hydrogen gas supplying apparatus (an apparatus for production of a hydrogen gas-containing gas), a hydrogen adding device (an apparatus for production of hydrogen water) or a non-destructive hydrogen incorporating device (for example an apparatus for non-destructively adding hydrogen gas to the inside of a bag for a biologically applicable liquid such as a drip-feed solution) in administration.

The hydrogen gas supplying apparatus ensures that hydrogen gas generated by reaction of a hydrogen generator (for example metallic aluminum or magnesium hydride) with water can be mixed with a diluting gas (for example air or oxygen) at a predetermined ratio (Japanese Patent No. 5228142). Alternatively, hydrogen gas generated by employing electrolysis of water is mixed with a diluting gas such as oxygen or air (Japanese Patent No. 5502973, Japanese Patent No. 5900688 or the like). In this way, a hydrogen gas-containing gas having a hydrogen concentration within the range of 0.5 to 18.5% by volume can be prepared.

The hydrogen adding device is an apparatus in which hydrogen is generated using a hydrogen generator and a pH adjustor, and dissolved in a biologically applicable liquid such as water (Japanese Patent No. 4756102, Japanese Patent No. 4652479, Japanese Patent No. 4950352, Japanese Patent No. 6159462, Japanese Patent No. 6170605, Japanese Patent Laid-Open No. 2017-104842 or the like). The combination of a hydrogen generator and a pH adjustor is, for example, a combination of metallic magnesium and a strongly acidic ion-exchange resin or an organic acid (for example malic acid or citric acid), a combination of metallic aluminum powder and calcium hydroxide powder. In this way, a hydrogen-dissolved liquid having a dissolved hydrogen concentration of about 1 to 10 ppm can be prepared (for example, trade name “7 Water” (QUASIA, Osaka, Japan), or the like).

The non-destructive hydrogen incorporating device is an apparatus or device in which hydrogen molecules are added to a commercially available biologically applicable liquid (for example, encapsulated in a hydrogen-permeable plastic bag such as a polyethylene bag) such as a drip-feed solution from the outside of a package, and such an apparatus or device is commercially available from, for example, MiZ Company Limited (Kanagawa, Japan) (www.e-miz.co.jp/technology.html). In this apparatus, a bag containing a biologically applicable liquid is immersed in saturated hydrogen water so that the bag is permeated with hydrogen, whereby hydrogen can be aseptically dissolved in the biologically applicable liquid until reaching equilibrium. The apparatus includes, for example, an electrolytic bath and a water bath, and water in the water bath is circulated through the electrolytic bath and the water bath, so that hydrogen can be produced by electrolysis. Alternatively, a simplified disposable device can be used for the same purpose (Japanese Patent Laid-Open No. 2016-112562, or the like). This device includes a biologically applicable liquid-containing plastic bag (a hydrogen-permeable bag, for example a polyethylene bag) and a hydrogen generator (for example metallic calcium, metallic magnesium/cation-exchange resin or the like) in an aluminum bag, and the hydrogen generator is covered with, for example, a nonwoven fabric (for example water vapor-permeable nonwoven fabric). The hydrogen generator covered with a nonwoven fabric is wetted with a small amount of water such as water vapor to generate hydrogen, and the plastic bag is permeated with the hydrogen and the hydrogen is non-destructively and aseptically dissolved in the biologically applicable liquid.

A hydrogen gas-containing gas or a hydrogen saturated biologically applicable liquid (for example water (for example purified water or sterilized water), physiological saline, or drip-feed solution), which is prepared using the above-described apparatus or device, can be orally or parenterally administered to a subject with cancer.

Another form of the composition of the present invention includes a hydrogen generator-containing dosage form (for example, a tablet or a capsule) which is prepared so as to be orally administered to (or ingested in) a subject and which enables hydrogen to be generated in the gastrointestinal tract. Preferably, the hydrogen generator is constituted by components approved as, for example, food or food additives.

### 3. Improvement of QOL by Suppression or Reduction of Symptoms Such as Cancerous Pain

A second aspect of the present invention provides a method for improving QOL in a subject with cancer, the method comprising administering the composition of the present invention to the subject with cancer.

The composition of the present invention provides improvement of the following symptoms, that is, an antitumor effect, for example a cancer cell growth-suppressive effect (or action), a terminal cancer patient life-extending effect (or action) and the like, and/or improvement of QOL (quality of life) selected from the group consisting of improvement of decreased appetite, improvement of insomnia, improvement of physical fatigue, and improvement of poor complexion or coloring in a subject with terminal cancer, in addition to suppression or reduction of cancerous pain (for example, Example 1 described later). Therefore, the method of the present invention can be used for improving, suppressing or reducing at least one of the above-described symptoms in a subject with cancer.

Cancerous pain observed in a terminal cancer patient significantly degrades QOL of the patient. By suppressing or reducing the cancerous pain by administration of the composition of the present invention, for example, decreased appetite can be improved to recover appetite, the state of insomnia can be improved, and physical fatigue or poor complexion or coloring (also referred to as blood flow or blood circulation) caused by bad conditions associated with cancer can be improved.

The method for administering the composition of the present invention to a subject is preferably pulmonary administration by, for example, inhalation or breathing when hydrogen gas is an effective ingredient, and is preferably by oral administration or intravenous administration (including drip infusion) when a hydrogen-dissolved liquid is an effective ingredient. In inhalation of the gas, the gas can be inhaled from the mouth or the nose through a nasal canula or a mask-type device covering the mouth and the nose, sent to the lung, and delivered to all parts of the body through blood.

For the hydrogen-dissolved liquid to be orally administered, the liquid cooled by storing the liquid preferably at a low temperature, or the liquid stored at normal temperature may be administered to a subject. It is known that hydrogen is soluble in water at a concentration of about 1.6 ppm (1.6 mg/L) at normal temperature and normal pressure, and the temperature-dependent variation of the solubility of hydrogen is relatively small. Alternatively, when the hydrogen-dissolved liquid is in the form of, for example, a hydrogen gas-containing drip-feed solution or injection solution prepared using the non-destructive hydrogen incorporating device, the hydrogen-dissolved liquid may be administered through parenteral administration such as intravenous administration or intraarterial administration to the subject.

A hydrogen gas-containing gas having the above-described hydrogen concentration or a hydrogen-dissolved liquid having the above-described hydrogen concentration can be administered once or two or more times (for example two or three times) per day over a period of 1 week to three months or more, for example 1 week to 6 months or more, to the subject. When the hydrogen gas-containing gas is administered, the hydrogen gas-containing gas can be administered, for example, for 10 minutes to 2 hours or more, 20 minutes to 40 minutes or more, or 30 minutes to 2 hours or more per administration. In addition, when the hydrogen gas-containing gas is administered through pulmonary administration by inhalation or breathing, the hydrogen gas-containing gas can be administered in an environment at atmospheric pressure, or an environment, for example, at a high pressure within the range of above standard atmospheric pressure (about 1.013 atm) and not more than 7.0 atm, for example at a high pressure within the range of 1.02 to 7.0 atm, preferably 1.02 to 5.0 atm, more preferably 1.02 to 4.0 atm, still more preferably 1.02 to 1.35 atm to the subject. By administration in an environment at a high pressure, systemic absorption of hydrogen in the subject can be promoted.

The environment at a high pressure can be formed by use of a high-pressure housing (for example a capsule-shaped housing) designed to have a sufficient strength so that for example, the hydrogen gas-containing gas (for example hydrogen-containing oxygen or air) can be internally injected to produce a high pressure of above standard atmospheric pressure and not more than 7.0 atm in the housing. Preferably, the shape of the high-pressure housing is generally free from sharp edges and rounded because the housing has pressure resistance. Preferably, the material of the high-pressure housing has a small weight and a high strength, and examples of the material include reinforced plastics, carbon fiber composite materials, titanium alloys and aluminum alloys. In the high-pressure housing, the subject can receive a composition for suppressing or reducing cancerous pain, the composition containing hydrogen gas together with oxygen gas or air.

The term “subject” as used herein includes mammals, for example primates including humans, companion animals such as dogs and cats, and fancy animals in zoos and the like. The subject is preferably a human.

In treatment of the symptoms such as cancerous pain, that is, improvement of QOL of a subject with cancer, with the composition of the present invention, it is desirable to use a hydrogen gas producing apparatus, a hydrogen water producing apparatus or a hydrogen gas adding apparatus (for example the above-described hydrogen gas supplying apparatus (or gaseous hydrogen inhalation apparatus), a hydrogen adding device (or hydrogen water producing apparatus) or a non-destructive hydrogen incorporating device (apparatus for non-destructively dissolving hydrogen gas in a biologically applicable liquid such as a drip-feed solution encapsulated in a hydrogen-permeable bag) which has been confirmed to have a sufficient treatment effect and sufficient safety.

### EXAMPLES

The present invention will be described in further detail by way of Examples below, but the technical scope of the present invention is not limited to these Examples.

### Example 1

### Improvement of QOL Including Suppression of Cancerous Pain in Terminal Cancer Patient by Breathing of Hydrogen Gas

### <Case 1>

A patient with terminal systemic metastatic cancer (70-year-old male) breathed a mixed gas of hydrogen and air using a gaseous hydrogen inhalation apparatus (MHG-2000α (registered trademark); MiZ Company Limited, Kanagawa, Japan) total 3 times for about 2 hours each time over about 1 month before death of the patient. The hydrogen concentration in MHG-2000α is about 6 to 7% by volume (hydrogen generation rate: about 140 ml/min). Before the breathing, the patient had severe cancerous pain, decreased appetite and poor complexion. However, due to the breathing of hydrogen gas, cancerous pain was reduced, appetite was recovered, and complexion was improved. During this period, the patient did not undergo treatment with opioid, and did not complain about pain.

### <Case 2>

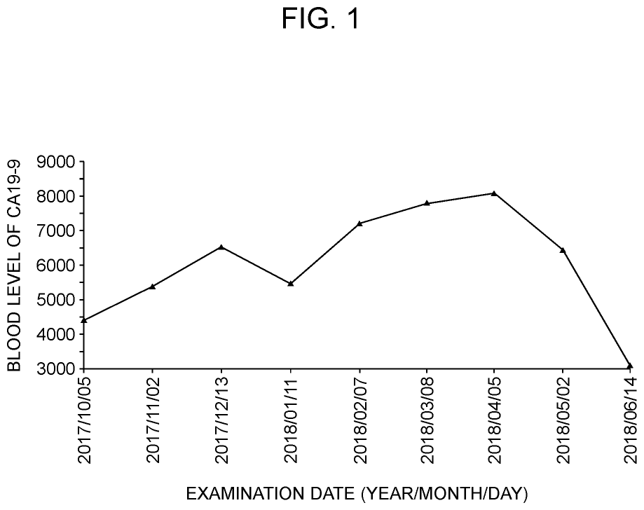

A patient with stage 4 terminal pancreatic cancer (81-year-old female) breathed a mixed gas of hydrogen and air using a gaseous hydrogen inhalation apparatus (MHG-2000α (registered trademark); MiZ Company Limited) for about 1 to 3 hours per day over about 8 months. The hydrogen concentration in MHG-2000α is about 6 to 7% by volume (hydrogen generation rate: about 140 ml/min). During this period, the patient underwent CT imaging examination once a month, and the result showed that there was no tendency of growth of cancer. This indicates that the breathing of hydrogen gas exhibited a cancer cell growth-suppressive effect. Further, from the result of examining the blood level of CA19-9 which is a pancreatic cancer marker (FIG. 1), it is apparent that the CA19-9 level sharply decreased about 6 months after the breathing of hydrogen gas. The patient hardly complained about physical fatigue after the first breathing. Further, before the breathing of hydrogen gas, the patient was told by the doctor that she would have 3 months to live, but even 9 months after the patient was told about the life expectancy, she was alive owing to breathing of hydrogen gas.

### <Case 3>

A patient with terminal pancreatic cancer (79-year-old male) breathed a mixed gas of hydrogen and air using a gaseous hydrogen inhalation apparatus (MHG-2000α (registered trademark); MiZ Company Limited) over about 4 months. Here, the mixed gas of hydrogen and air was breathed twice a week (duration each time: 1 to 3 hours) over first 2 months and for about 24 hours per day over subsequent 2 months. The hydrogen concentration in MHG-2000α is about 6 to 7% by volume (hydrogen generation rate: about 140 ml/min). The patient died about 40 days after breathing of the mixed gas of hydrogen and air was discontinued, and during this period, the patient did not undergo treatment with an opioid. Before the breathing of hydrogen, the patient suffered from cancerous pain, and continued to have insomnia, but after the breathing of hydrogen gas, the patient did not complain about pain or oppression, improved in insomnia, had appetite, and was well conscious. The patient put a smile back on his face in conversation with the family in everyday life.

### Industrial Applicability

According to the present invention, symptoms caused by cancer, such as cancerous pain, can be significantly suppressed or reduced and a cancer cell growth-suppressive effect and a life-extending effect can be provided simply by administering molecular hydrogen to a cancer patient. Hydrogen itself has no known side effect, so that QOL of the patient can be improved in cancer treatment.

1. A composition for improvement of QOL including improvement, suppression or reduction of at least one symptom attributed to cancer selected from the group consisting of cancerous pain, decreased appetite, insomnia, physical fatigue, and poor complexion or coloring in a subject with cancer, the composition comprising molecular hydrogen as an effective ingredient.

2. The composition according to claim 1, wherein the composition is in the form of a hydrogen gas-containing gas and/or a hydrogen-dissolved liquid.

3. The composition according to claim 2, wherein a hydrogen concentration of the hydrogen gas-containing gas is 0.5 to 18.5% by volume.

4. The composition according to claim 2, wherein a hydrogen concentration of the hydrogen-dissolved liquid is 1 to 12 ppm.

5. The composition according to claim 1, wherein the composition is administered to the subject by pulmonary administration, intravenous administration or oral administration.

6. The composition according to claim 5, wherein the pulmonary administration is performed in an environment at atmospheric pressure, or in an environment at a high pressure of 1.02 to 7.0 atm.

7. The composition according to claim 1, wherein the composition is prepared in situ using a hydrogen gas producing apparatus, a hydrogen water producing apparatus or a hydrogen gas adding apparatus, in administration of the composition to the subject.

8. The composition according to claim 1, wherein the subject is a human.

9. The composition according to claim 1, wherein the subject has terminal cancer.

10. The composition according to claim 1, wherein the composition further has cancer cell growth-suppressive action and life-extending action.

11. A method for improving QOL in a subject with cancer, the method comprising administering the composition according to claim 1 to the subject with cancer.
